(19) United States
(12) Patent Application Publication
(10) Pub. No.: US 2011/0172624 A1
(43) Pub. Date: Jul. 14, 2011

(54) USE OF LYOCELL FIBERS AS WELL AS ARTICLES CONTAINING LYOCELL FIBERS

(75) Inventor: Sigrid Redlinger, Lenzing (AT)

(73) Assignee: LENZING AKTIENGESELLSCHAFT, Lenzing (AT)

(21) Appl. No.: 13/119,828

(22) PCT Filed: Sep. 21, 2009
(86) PCT No.: PCT/EP2009/062177

§ 371 Date: Mar. 18, 2011

(30) Foreign Application Priority Data
Sep. 22, 2008 (AT) A 1471/2008
Nov. 11, 2008 (AT) A 1750/2008

(51) Int. Cl. A61L 15/28

(52) U.S. Cl. 604/375

(57) ABSTRACT

The invention relates to the use of non-modified Lyocell fibers, a yarn containing non-modified Lyocell fibers, a textile fabric containing non-modified Lyocell fibers or a textile article containing non-modified Lyocell fibers as a product for in particular dry wound care.

### BACKGROUND OF THE INVENTION

### Field of the Invention

The invention relates to the use of Lyocell fibers, a yarn containing Lyocell fibers, a textile fabric containing Lyocell fibers or a textile article containing Lyocell fibers.

The generic name “Lyocell” was issued by the BISFA (The International Bureau for the Standardisation of Man Made Fibers) and stands for cellulose fibers, which are prepared from cellulose solutions in an organic solvent. Solvents preferably used are tertiary amine oxides, in particular N-methyl-morpholine-N-oxide (NMMO). A method for preparing Lyocell fibers is e.g. described in U.S. Pat. No. 4,246,221.

The present invention relates in particular to the use of Lyocell fibers in products for wound care.

The expert in general distinguishes three phases of the physiological wound care:

1) Coagulation and Inflammation:

In this phase, the primary aim of the treatment is the removal of cell debris and micro-organisms as well the formation of a provisional matrix.

2) Proliferation

Here, the primary aim is the formation of granulation tissue and the re-epithelialisation for the formation of a temporary barrier.

3) Scarring (Repair)

Herein, there is attempted to reconstitute the functionality of the skin as completely as possible (complete barrier, tear strength and sensitivity).

In the field of wound care, the expert distinguishes between conventional dry and moist wound care. Moist wound care methods aim at conserving a moist wound environment in the treatment of chronic wounds. Dry wound care in the case of acute wounds aims at protecting against infections and adsorbing wound secretion. The present invention relates to a product for dry wound care, in the case of which presently there is primarily used cotton.

As substrates for wound care products there are, on the one side, used essentially untreated products such as, for example, bleached cotton, which have as little decelerating influence on wound healing as possible. In theory, wounds would heal most quickly (in a sterile and moist environment) without application of any bandage or the like.

### Description of Related Art

On the other side, there are a great variety of products, which concretely promote wound healing due to their specific characteristics. Among these, there are to be mentioned, of course, therapeutically active substances, but also fibers modified with wound healing promoting agents, such as e.g. alginates, chitosans or carboxymethyl groups.

WO 94/16746 as well as DE 100 09 248 describe the use of Lyocell fibers, which have been modified with carboxymethyl groups (CMC groups), in wound healing products.

WO 2005/026424 A1 describes the use of Lyocell fibers for the treatment of textile contact sensitivity or skin diseases, in particular xerosis, atopic eczema or psoriasis.

In the Austrian patent application A 1471/2008 there are described cellulose fibers modified with chitosan and their use as a wound healing product. In a comparative test, there were found significantly more proliferative cells at the wound edge and in tendency more cells in the regenerating epidermis with Lyocell fibers that have been modified with chitosan than with a non-treated Lyocell fibre and cotton.

DE 100 07 794 A1, DE 100 37 983 A1 and EP 1 354 914 describe polymer compositions comprising respectively a biologically degradable polymer and various modified agents such as algae, alkaloids and herbs. The polymer composition may be provided in the form of fibers, which are spun from, for example, a mixture of the modifying agent with a cellulose solution in a tertiary amine oxide.

Surprisingly, there has been found out that non-modified Lyocell fibers (this is Lyocell fibers containing no modifying active substances like chitosan, algae, alkaloids or herbs and not being provided in the form of a derivative, such as e.g. in carboxymethylated form) exert a positive influence on wound healing and the morphology of a wound edge.

### SUMMARY OF THE INVENTION

Accordingly, the present invention relates in a first aspect to non-modified Lyocell fibers, a yarn containing non-modified Lyocell fibers, a textile fabric containing non-modified Lyocell fibers or a textile article containing non-modified Lyocell fibers, respectively for the specific use as a wound care product, in particular for dry wound care.

In another aspect the present invention relates to a product for dry wound care, characterized in that it contains, in its part intended for application onto the wound, non-modified Lyocell fibers.

The present invention, hence, relates in general to the use of non-modified Lyocell fibers for the preparation of a wound care product.

There has been found that non-modified Lyocell fibers significantly accelerate wound healing in comparison to other non-modified products that have so far been used as substrates in dry wound care (i.e. products containing no active substances or wound healing promoting agents), in particular in comparison with bleached cotton.

In particular, in the examination of textile articles containing non-modified Lyocell fibers, there was found, in the porcine ex-vivo wound model according to the teaching of WO 2004/092726 and WO 2004/092354, that articles containing non-modified Lyocell fibers, in comparison to bleached cotton and polyester, have a positive influence on the wound healing progress and the morphology of the wound edge.

The possible use of a non-modified Lyocell fiber instead of or in addition to rather expensive modified products such as CMC fibers or fibers modified with chitosan naturally presents a huge financial advantage.

Preferably, the yarn, the textile fabric and the textile article, respectively, used according to the invention are essentially completely consisting of non-modified Lyocell fibers.

Alternatively, the yarn, the textile fabric and the textile article, respectively, used according to the invention may contain foreign fibers in an amount of up to 90%, preferably 10 to 50%.

“Foreign fibers”, for the purpose of the present invention, are fibers that differ than non-modified Lyocell fibers, for example cotton, polyester, viscose and modal fibers but also modified Lyocell fibers. Preferably there are also used non-modified fibers as foreign fibers, this is fibers that are not modified with active substances or derivatized, etc.

### DETAILED DESCRIPTION OF THE INVENTION

A textile fabric for the use according to the invention may especially be provided in the form of a woven or knitted or non-woven fabric.

As already mentioned, the invention relates in another aspect to a wound care product, in particular for dry wound care, characterized in that it contains, in its part intended for application onto the wound, non-modified Lyocell fibers.

The product according to the invention may be selected from a group consisting of patches, wound paddings, bandages, bandaging material, padding fabrics for plasters, underwear, nightwear for individuals suffering from neurodermatitis, orthopaedic supporting bandages, supporting bandages, wound gaze, compresses, medical gauze, compression bandages, compression stockings and tube bandages.

The wound care product according to the invention is preferably characterized in that the portion of non-modified Lyocell fibers in the part intended for application onto the wound is at least 10%, preferably 50 to 100% and particularly preferably 90 to 100%.

The other fibers in the part intended for application on to the wound may, as mentioned above, be foreign fibers, in particular non-modified foreign fibers.

The Lyocell fiber used according to the invention may be provided in a titer range usual for textile products or non-woven fabrics and in a common cutting length, respectively. The fiber may typically have a titer of 1.3 dtex and a cutting length of 38 mm. The fiber may be used in bleached form.

### EXAMPLES

In a test of knitted fabrics, prepared from 100% non-modified Lyocell fibers (titer 1.3 dtex, cutting. length 38 mm) there was found in the porcine ex-vivo wound model according to WO 2004/092726 and WO 2004/092354 that knitted fabric of 100% Lyocell has a positive influence on wound healing and the morphology of the wound edge in comparison to bleached cotton and polyester. In particular in comparison with bleached cotton, non-modified Lyocell fibers have a significantly better effect (statistical evaluation of respective 10 models with the aid of a paired student T-test p less than or equal to 0.5).

### Skin Organ Culture Model—Wound Healing Model

From the plicae of washed and sterilized pig ears, there were taken punches with a diameter of 6 mm. From the middle of the punches, there were removed the epidermis and the upper dermis in an area of 3 mm. Subsequently, the models were incubated in an air-liquid interphase at 37° C., 5% CO₂ and saturated humidity in the dermatological clinic. Immediately upon generation, the models were applied in the control 5 μl PBS. For the other samples, there were taken knitted fabric samples with a diameter of 4 mm using biopsy punch platelets and applied onto the wounds.

After 48 h the models were shock-frozen and stored at −80° C.

### Histochemistry: Preparation of Model Sections

The models were completely embedded in tissue freezing medium (company Leica, Nussloch), and there were generated cryostat sections with a width of 6 μm. Special attention was paid to the positioning of the respective model in the middle of the arrangement. The sections were placed on SuperFrost slides, air-dried, fixed for 10 min in −20° C. cold acetone and stored at −80° C.

HE staining (general morphology): All models were stained with haematoxylin/eosin (HE) (2 sections each). The stainings were then evaluated under a Leica light microscope DM LS and an Olympus Camedia digital camera.

### Evaluation Parameter: Wound Healing Progress and Morphology—Good/Bad Conservation of the Wound Edge (Maceration)

The wound healing progress is distinguished into the following categories
0: no wound healing progress
1: small wound tissue
2: big wound tissue
3: closed sheet
4: multi-layered closed sheet

The evaluation of the wound healing progress is performed on the basis of the PBS control in order to take into account the wound healing potential of the respective pig, evaluated by means of the values obtained using PBS (physiological phosphate puffer). In general there may always be observed that wound healing decelerates if wound paddings are applied to the wound. The wound healing progress obtained with only PBS, hence, is the maximum value (ideal wound healing) that may be obtained (100% or value 1) in the skin organ culture model.

Surprisingly, there was evaluated for knitted fabrics made from non-modified Lyocell fibers a mean value of 0.8 for the wound healing progress standardized on the PBS control, with in part even obtaining individual values of >1. With knitted fabrics made from bleached cotton, there may only be obtained mean values of 0.3, with polyester fibers of 0.6.

The results are summarized in the following table:

TABLE

| | | | | | | | | | | | | Mean | |
|---|---|---|---|---|---|---|---|---|---|---|---|---|---|
| Sample | 457 | 458 | 459 | 463 | 464 | 465 | 466 | 467 | 468 | 469 | n | values | SEM |
| 1 | | 0.23 | | 0.44 | | 0.51 | | 0.78 | | 1.03 | 5 | 0.60 | 0.15 |
| 2 | 0.50 | 0.71 | 0.45 | | 0.79 | 1.36 | | 0.71 | 0.21 | 1.71 | 8 | 0.81 | 0.18 |
| 3 | 0.00 | 0.11 | 0.15 | 0.36 | | 0.96 | 0.00 | 0.56 | 0.11 | 0.06 | 8 | 0.26 | 0.13 |
| 4 | 1.00 | 1.00 | 1.00 | 1.00 | 1.00 | 1.00 | 1.00 | 1.00 | 1.00 | 1.00 | 10 | 1.00 | 0.00 |

Sample 1: polyester fibre (Tergal PES)
Sample 2: non-modified Lyocell fiber
Sample 3: bleached cotton
Sample 4: PBS control

Ideal wound padding should not only promote wound healing but rather also not affect the wound edge. For this reason, there is to be taken care to conserve a good morphology of the wound edge.

When using non-modified Lyocell fibers there was observed a good morphology of the wound edge. Cotton, on the other hand, showed a bad conservation of the wound edge.

What is claimed is:

1. Non-modified Lyocell fibers, a yarn containing non-modified Lyocell fibers, a textile fabric containing non-modified Lyocell fibers or a textile article containing non-modified Lyocell fibers for the specific use as a product for wound care, in particular for dry wound care.

2. A yarn, textile fabric or textile article for the specific use according to claim 1, each essentially completely consisting of non-modified Lyocell fibers.

3. A yarn, textile fabric or textile article for the specific use according to claim 1, each containing foreign fibers in an amount of up to 90%, preferably 10 to 50%.

4. A textile fabric for the specific use according to any of the claims 1 to 3 in the form of a woven or knitted or non-woven fabric.

5. A product for wound care, in particular for dry wound care, characterized in that it contains, in its part intended for application onto the wound, non-modified Lyocell fibers.

6. A product according to claim 5, characterized in that it is selected from a group consisting of patches, wound paddings, bandages, bandaging material, padding fabrics for plasters, underwear, nightwear for individuals suffering from neurodermatitis, orthopaedic supporting bandages, supporting bandages, wound gaze, compresses, medical gauze, compression bandages, compression stockings and tube bandages.

7. A product according to claim 5 or 6, characterized in that the portion of the non-modified Lyocell fibers in the part intended for application onto the wound is at least 10%, preferably 50 to 100%, particularly preferably 90 to 100%.
